(19) United States
(12) Patent Application Publication
(10) Pub. No.: US 2014/0365142 A1
(43) Pub. Date: Dec. 11, 2014

(54) Wearable Blood Alcohol Measuring Device

(71) Applicant: James Armand Baldwin, Palo Alto, CA (US)

(72) Inventor: James Armand Baldwin, Palo Alto, CA (US)

(73) Assignee: ANTIMATTER RESEARCH, INC., Palo Alto, CA (US)

(21) Appl. No.: 14/298,847

(22) Filed: Jun. 6, 2014

Related U.S. Application Data
(60) Provisional application No. 61/833,469, filed on Jun. 11, 2013.

(51) Int. Cl. G01N 33/98; B60K 28/06; A61B 5/00

(52) U.S. Cl. CPC G01N 33/98; A61B 5/683; A61B 5/0002; B60K 28/066
USPC 702/24

(57) ABSTRACT

A system and method of measuring and analyzing a user's blood alcohol content are provided, the system comprising a wearable alcohol sensor that measures a user's blood alcohol content continuously or at random intervals, and a mobile device that receives blood alcohol content data from the wearable alcohol sensor, said mobile device being able to store and analyze the blood alcohol content data, to warn the user or others when the user's blood alcohol content is too high or when the user is unable to drive legally, and to use the stored data and the analysis results to predict the user's future blood alcohol content.

### CROSS-REFERENCE TO RELATED APPLICATIONS

The present application takes priority from Provisional Patent App. No. 61/833,469, filed Jun. 11, 2013, which is incorporated herein by reference.

### FIELD OF THE INVENTION

The present invention generally relates to a blood alcohol monitoring system, and more particularly, relates to a non-invasive, wearable apparatus and method for continuous and discreet monitoring and prediction of blood alcohol levels.

### BACKGROUND

Individuals desiring to consume alcoholic beverages recreationally may need to monitor their alcohol intake as they are consuming the beverages to avoid serious problems. For example, someone who wishes to drive after consuming alcohol may need to ensure that they do not exceed the maximum legal limit. Someone may wish to consume enough alcoholic beverages to enjoy a pleasant state of inebriation while avoiding a blackout state. Someone may wish to moderate their alcohol intake to avoid alcoholism. Since it is often difficult to determine one's exact degree of inebriation, especially while inebriated, many people find it difficult to titrate their alcohol consumption accordingly.

The most commonly used devices for measuring inebriation are breath-testing analyzers. Such devices are available for consumers as well as for law enforcement use. However, breathalyzers have several serious problems. First of all, they are not discreet—a consumer wishing to measure their blood alcohol content must blow into a highly conspicuous device. This may make some users self-conscious to the point that they would be unwilling to use such a device, especially when inebriated at a social gathering. The use of a breathalyzer may carry a further stigma considering the use of such devices for law enforcement purposes. Secondly, the more inebriated a user is, the less likely they are to remember to use a breathalyzer to measure their degree of inebriation. Thirdly, since traces of alcohol remain in the mouth after drinking, a breathalyzer is likely to be inaccurate and to overestimate a user's alcohol content. Finally, since the user's blood alcohol content is measured only at a few discrete times, it is difficult to make any sort of prediction of future intake or to help the user titrate their intake.

Transdermal alcohol sensors measure the amount of alcohol excreted by the user's skin. As a person ingests alcohol, most of the alcohol is metabolized by the liver, but some of it is excreted with perspiration by the skin; the average person emits approximately one liter of skin perspiration a day. The amount of alcohol in the perspiration is correlated to blood alcohol content. A transdermal alcohol sensor is typically a device that generates electricity in the presence of ethanol; putting such a device on the skin allows an estimate to be made of the amount of ethanol excreted by the skin. The advantage of a transdermal alcohol sensor is that measurements can be made continuously and are not dependent on the user's remembering to use the device; furthermore, a transdermal alcohol sensor is more discreet and easy to hide under one's clothing.

Transdermal alcohol sensors are used in law enforcement when an individual's alcohol intake needs to be monitored by the authorities. However, they are typically not available for the consumer market, and law enforcement devices using transdermal alcohol sensors typically do not offer features that enable a consumer to easily interact with the device, use it to predict their blood alcohol content at a point in the future, or use it to titrate their alcohol intake.

A need exists for an alcohol sensor device that is discreet and that allows a consumer to predict their blood alcohol content at some future point, titrate their alcohol intake, and easily see their current blood alcohol content.

### BRIEF DESCRIPTION OF THE DRAWINGS

FIG. 1 shows a diagram of the preferred embodiment of the invention.

FIG. 2 shows a preferred embodiment of the wearable sensor module worn by a user.

FIG. 3 shows a transdermal alcohol sensor used in the preferred embodiment of the invention.

FIG. 4 shows a screenshot from the app used in the preferred embodiment of the invention.

FIG. 5 shows a screenshot from the app used in the preferred embodiment of the invention.

FIG. 6 shows a screenshot from the app used in the preferred embodiment of the invention.

FIG. 7 shows a screenshot from the app used in the preferred embodiment of the invention.

### SUMMARY OF THE INVENTION

An object of the present invention is to provide a system and method for continuously measuring a user's blood alcohol content and displaying the measurement for the user or for another person or entity.

Another object of the present invention is to provide a discreet way to measure and display a user's blood alcohol content that does not attract undue attention or stigma.

Another object of the present invention is to provide a system and method for predicting a user's blood alcohol content at a future point.

Another object of the present invention is to provide a system and method for modeling a user's future blood alcohol content depending on intended consumption and guiding the user in their alcohol consumption.

In its preferred embodiment, the system of the present invention comprises a wearable alcohol sensor that measures a user's blood alcohol content, preferably by measuring the amount of alcohol in the user's insensible perspiration. The wearable alcohol sensor also comprises a mounting attachment that attaches the wearable alcohol sensor to the user's body, and a communication module that communicates with a mobile device such as a smartphone, a tablet, a computer, or any other similar device. The mobile device receives data from the wearable alcohol sensor, processes that data, and presents the results to the user.

The mobile device preferably comprises an app that interprets data received from the wearable alcohol sensor. Such an app can display a user's past blood alcohol history for a given period of time, display a user's current blood alcohol reading, or predict a user's future blood alcohol content. The prediction can be made based on several different assumptions—for example, if the user consumes no other alcoholic drinks for the rest of the night, if the user continues drinking at the same rate, if the user consumes a certain number of drinks of a certain type, and so on.

In an embodiment, the mobile device can warn a user when their blood alcohol content reaches a preset maximum value, and can cause the mobile device to sound an audible or visible alarm, vibrate, send an email or a text message, or initiate a phone call. This would inform the user that their blood alcohol content is too high without stigmatizing them at a party.

In an embodiment, the mobile device can also warn other people when a user's blood alcohol content reaches a preset maximum value. The warning can be effectuated by a phone call, a text message, an email, or any other means of communication.

In an embodiment, the mobile device can take a photo of a user's surroundings if a user's blood alcohol content reaches a preset maximum value, and send the photo to other people. This may be used by a user's friends or family to find the user when a user is too inebriated to be safely left alone.

In an embodiment, the system of the present invention can predict a time at which a user may legally drive a motor vehicle if a user ingests no further alcohol. This may assist a user in deciding whether or not to take another drink at a party, or in deciding when to go home from a party. If a user is too inebriated to be able to legally drive the same day, the mobile device may communicate with others, such as a user's friends or family, or a taxi company.

In making these predictions, the system of the present invention may use past blood alcohol content history, a user's height, weight, gender, and race, or both, to estimate the user's rate of alcohol metabolism. The system may “learn” the user's rate of alcohol metabolism over time.

Since skin-based alcohol sensors have a lag time of about 15 minutes between the time an alcoholic drink is ingested and the time it is excreted by the skin, the system may have a secondary sensor to determine when a user is consuming an alcoholic drink. Such a sensor may use a user's motion pattern (i.e. raising a glass to their lips), a visual image of a glass in the user's hand, the scent of alcohol, or any other commonly known phenomena to identify when a user consumes an alcoholic drink. A user may also be able to enter manually into the system when he or she consumes an alcoholic drink.

The method of the present invention comprises continuously measuring a user's blood alcohol content by a wearable sensor, transmitting data from the sensor to a mobile device, and using the mobile device to analyze the data and to make predictions using the data.

### DETAILED DESCRIPTION OF THE PREFERRED EMBODIMENT

The present invention now is described more fully hereinafter with reference to the accompanying drawings, in which preferred embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

It will be understood that when an element is referred to as being “on” another element, it can be directly on the other element or intervening elements may also be present. In contrast, when an element is referred to as being “directly on” another element, there are no intervening elements present. It will be understood that when an element is referred to as being “connected” or “attached” to another element, it can be directly connected or attached to the other element or intervening elements may also be present. In contrast, when an element is referred to as being “directly connected” or “directly attached” to another element, there are no intervening elements present. The terms “upwardly”, “downwardly”, “vertical”, “horizontal” and the like are used herein for the purpose of explanation only.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. All publications, patent applications, patents, and other references mentioned herein are incorporated herein by reference in their entirety. As used herein, the term “and/or” includes any and all combinations of one or more of the associated listed items.

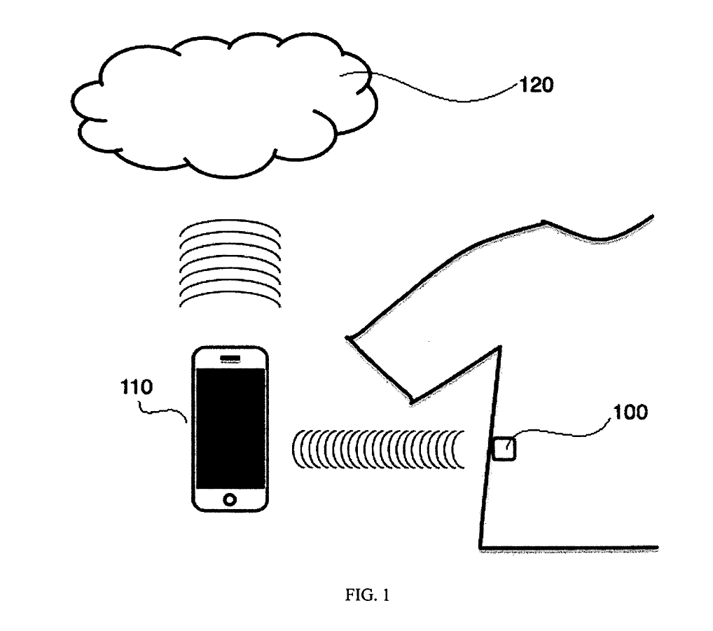

In its preferred embodiment, as shown in FIG. 1, the system of the present invention comprises a wearable sensor module 100 and a mobile device 110 (preferably a smartphone) comprising an app that enables the mobile device 110 to send data to, and to receive data from, the wearable sensor module 100. The wearable sensor module 100 preferably comprises an alcohol sensor and a communication interface. In its preferred form, the wearable sensor module 100 weighs an ounce or less and has a form factor and color that is unnoticeable under a user's clothes. The wearable sensor module 100 may be worn anywhere on the user's body as long as the alcohol sensor is able to be in direct connection with the user's skin. For example, it may be worn on the user's upper arm, on the user's ankle, tucked into a user's bra strap or underwear waistband, or adhered to any part of the user's body, preferably hidden under clothing for greater discretion. In various embodiments, the wearable sensor module 100 may comprise an elastic strap to enable a user to wear it around their upper arm or ankle, a clip to attach it to the user's waistband or a bra strap, or adhesive elements to adhere it directly to the user's skin. Any other attachment means known in the art of wearable device design can be used as long as the wearable sensor module 100 is in continuous and secure contact with the user's skin.

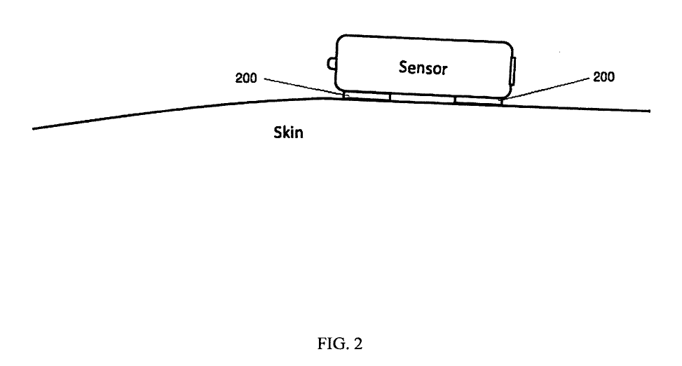

The wearable sensor module preferably comprises a dermal seal and spacer 200 that creates a seal between the user's skin and the alcohol sensor, as shown in FIG. 2. The dermal seal and spacer 200 may comprise a foam pad or pads of a predetermined thickness, rubber bellows, or other similar elements that maintain the wearable sensor module at a controlled distance from the user's skin and create a seal between the user's skin and the alcohol sensor. It is preferable for the wearable sensor module 100 and the dermal seal and spacer 200 to be non-allergenic and non-irritating to the skin.

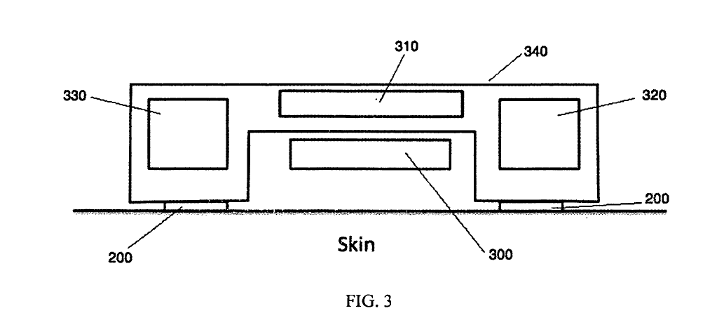

As shown in FIG. 3, the wearable sensor module preferably comprises a housing 340 that contains the alcohol sensor 300, the communications interface 310, a microprocessor 320, and a battery 330. A dermal seal and spacer 200 are affixed to the housing 340. The housing 340 is preferably made of plastic or any other material that is lightweight, non-allergenic, and that does not block wireless communication; it is preferably skin-colored to make it more discreet. In an embodiment, the housing 340 can also comprise one or more control buttons, such as an on/off button, a reset button, or a manual test button. In an embodiment, pressing the manual test button will preferably trigger the wearable sensor module to conduct an alcohol test on the user.

The wearable sensor module may also comprise trigger circuitry that triggers the alcohol sensor to test the user's blood alcohol content—preferably continuously, or at regular or irregular intervals. Such trigger circuitry may also trigger the communication module to transmit the test results to the mobile device. The trigger circuitry may be configurable by the user to operate in regular interval mode, irregular interval mode, or continuous mode; the user may also be able to set the intervals at which testing will be performed.

The alcohol sensor 300 is preferably any sensor that can measure a user's blood alcohol level continuously or at intermittent intervals without the user's active involvement. In the preferred embodiment, the alcohol sensor is a transdermal alcohol sensor that measures a user's blood alcohol level by detecting the amount of alcohol in the user's insensible perspiration. Such a sensor preferably comprises a fuel-cell module that generates electricity in the presence of alcohol.

In alternate embodiments, the alcohol sensor 300 may be an optical sensor that measures light absorption of the user's skin, or any other sensor capable of measuring a user's blood alcohol content continuously or at intermittent intervals.

The data generated by the alcohol sensor 300 is transmitted through the communications interface 310 to the mobile device 110, as shown in FIG. 1. The mobile device 110 is preferably a smartphone, but can be a tablet, a laptop, an mp3 player, or any other device that is capable of wireless communication and can be transported by a user to an occasion involving drinking. The alcohol sensor 300 preferably takes continuous readings and continuously communicates with the mobile device 110, but may also take readings at regular or irregular intervals and communicate with the mobile device at regular or irregular intervals. The communications interface is preferably Bluetooth, but may also be wi-fi, 3G or 4G, or any other wireless communications interface commonly used in the art. In an embodiment, the wearable sensor module may transmit its data to the cloud 120 and the mobile device may retrieve the data from the cloud 120.

The mobile device 110 preferably comprises a processor, a memory, and a communication interface, said memory comprising an app that enables data from the wearable sensor module to be sent to the mobile device via the communication interface, and enables commands to be sent to the wearable sensor module from the mobile device via the communication interface.

The app preferably performs at least the following functions. It enables the user to see the data from the blood alcohol sensor displayed on the screen of their mobile device; it triggers the wearable sensor module to measure the user's blood alcohol level; it displays historical data regarding the user's blood alcohol level; and it predicts the user's future blood alcohol levels.

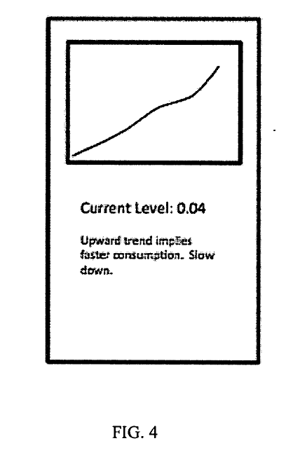

FIGS. 4-7 show several screenshots from the app. A user can find out their current blood alcohol content by simply activating the app and going to the measurement screen, which displays the current blood alcohol reading and a graph of historical blood alcohol consumption, as shown in FIG. 4. In an embodiment, the app can also display a warning when the alcohol consumption is increasing too fast, as shown in FIG. 4. In another embodiment (not shown), a user can find out their current blood alcohol level by activating the app on their mobile device and pressing the “Measure BAC” button. The app then triggers the mobile device to send a signal to the wearable sensor module, causing the wearable sensor module to either conduct a reading (if in intermittent mode) or to send the most current reading of the user's blood alcohol level to the mobile device (if in continuous mode) and display it. The BAC reading will then appear on the mobile device screen. Since the user does not have to perform any action other than pressing a button on their mobile device screen, and since people check their mobile devices for many different reasons, it will be possible for the user to measure their blood alcohol level without being stigmatized (in contrast, it is very difficult to hide the act of blowing into a breathalyzer at a party). The BAC reading may be displayed as a simple number, or it may be displayed in other ways (such as icons to show how many drinks the user has already had and how many more drinks the user may have before reaching a predetermined maximum level of inebriation, or colors to indicate various levels of inebriation). The user may set a predetermined maximum allowable level of inebriation ahead of time.

In an embodiment, the app can also store and display a user's history of blood alcohol levels as a graph, a data table, or as any other display. For example, a user may wish to see their history of alcohol consumption at a party, or over a week's time, as an inducement to greater moderation. FIG. 4 shows a sample display of a user's blood alcohol level history, and a warning given to a user whose alcohol consumption rate is increasing.

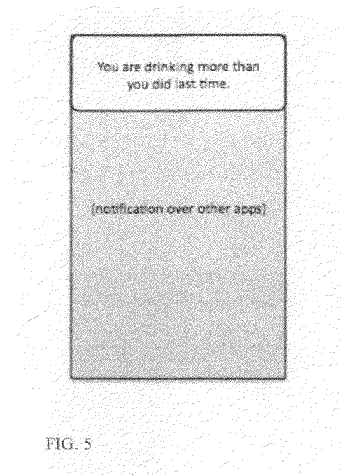

FIG. 5 shows a discreet notification from the app given to a user whose drinking is increasing. The push notification can appear over other apps if the user allows it to do so.

In an embodiment, the app uses the blood alcohol level history to calculate the rate at which the user metabolizes alcohol and to learn the user's drinking patterns. This enables the app to customize its operation to the individual user.

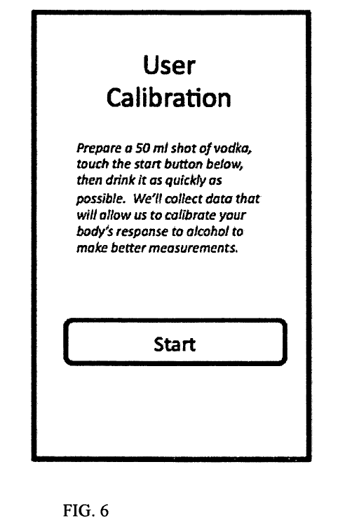

In an embodiment, the app comprises a prediction module that enables it to make predictions about the user's inebriation level depending on the user's individual alcohol metabolism. Since different people metabolize alcohol at different rates and react to alcohol differently, the prediction module preferably is able to learn the user's individual reactions. In an embodiment, the user can “train” the prediction module by consuming predetermined amounts of alcohol, entering the amounts into the system, and measuring their blood alcohol content by means of the wearable sensor module. The wearable sensor module can then learn how fast the user's body metabolizes the consumed alcohol and use it to make more accurate predictions. FIG. 6 shows a sample “training” screen. In another embodiment, the prediction module uses the user's height, weight, gender, and other relevant variables to predict the user's alcohol metabolism. In another embodiment, the prediction module uses historical data regarding the user's blood alcohol levels to estimate the level at which the user's body metabolizes alcohol. The prediction module is preferably able to estimate the user's future blood alcohol levels in various situations (i.e. if no more drinks are consumed, if the user keeps drinking at the same rate, if the user only has one more drink, and so on).

In an embodiment, the prediction module can warn the user when their inebriation level is likely to cause problems. For example, the prediction module can sound an alarm or cause the mobile device to vibrate when the user is dangerously close to a maximum predetermined level of inebriation. The alarm can be discreet to avoid stigmatizing the user. In an embodiment, the prediction module can trigger a text message to be sent to the user or a phone call to be made.

In an embodiment, the prediction module may also alert others when a user's inebriation level is likely to cause problems. For example, a woman going out drinking may set her mobile device to alert her friends when she reaches a dangerous level of inebriation so that they can help her avoid a dangerous situation. The mobile device may be triggered to send a standardized text message, an email, or any other type of message or alert. The mobile device may also be triggered to take a picture of the user's surroundings automatically when a maximum predetermined level of inebriation is reached and to send the picture to others to allow them to find the user easily.

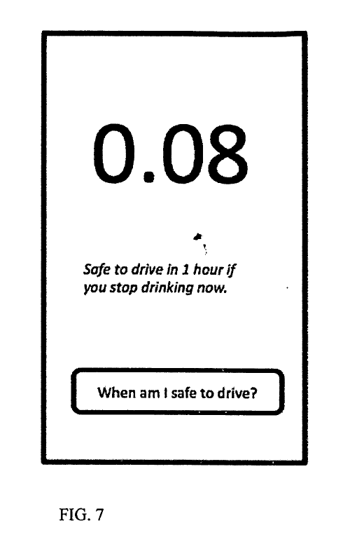

In an embodiment, the prediction module can instruct the user when they need to stop drinking so that they can still drive home safely. FIG. 7 shows a sample screen from the app, showing the user's current blood alcohol level and a prediction of how soon it will be safe to drive. The prediction module may use the user's current blood alcohol level as measured by the wearable sensor module and the estimated time of going home to determine what the user's blood alcohol level is going to be at the time of going home, assuming the user's typical metabolism rate is known. Alternately, the prediction module may display an estimated time at which it would be safe for the user to drive. In an alternate embodiment, the prediction module may cause the mobile device to contact the user's friends, family, or a taxi service when the user reaches a level of inebriation that would make it unsafe for them to drive home after a party at a reasonable time.

In an embodiment, the prediction module can model the user's future blood alcohol content depending on various hypothetical situations. For example, a user may wish to see if it is safe for them to consume a particular drink at a given time, followed by another type of drink at a different time. The prediction module may comprise a database of typical drinks and their alcohol content, and may enable the user to select the types of drinks and the times at which they are consumed and to see what their individual reaction to those drinks is likely to be. For example, the prediction module may determine that a user is likely to get blackout drunk after consuming three drinks of a particular type at a given time, or that it may be unsafe for a user to drive home at 1 am after consuming two drinks of a particular type at midnight.

Since a transdermal alcohol sensor has about a 15-minute lag time between consumption of alcohol and its excretion by the skin, the prediction module preferably comprises a way to compensate for the lag time by estimating the user's blood alcohol content after a 15-minute time interval has elapsed. In an embodiment, the system comprises another sensor that can determine when a drink is being ingested; for example, the wearable sensor module can comprise a second sensor that detects a glass in a user's hand, or detects a sound, scent, or other phenomenon associated with a user's consumption of alcohol. The second sensor can then communicate with the prediction module to alert it that the user is consuming another drink and to adjust its estimate of the user's current blood alcohol content. In another embodiment, the user may manually enter it into the app when they consume a drink. The system can then use the entered data to adjust its predictions of the user's blood alcohol content.

As has been mentioned previously herein, many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

1. A system for measuring blood alcohol levels in a user, comprising:
a wearable alcohol sensor, said wearable alcohol sensor capable of measuring a user's blood alcohol levels continuously and noninvasively, said wearable alcohol sensor comprising:
a mounting attachment for attaching the wearable alcohol sensor to the user's body;
a communication module capable of wireless communication;
a mobile device, comprising a processor, a memory, and a communication module capable of wireless communication, wherein the processor is configured to receive data from the wearable alcohol sensor, process data received from the wearable alcohol sensor, and to present processed data to the user.

2. The system of claim 1, wherein the wearable alcohol sensor measures a user's blood alcohol level by detecting an amount of alcohol in the user's insensible perspiration.

3. The system of claim 1, wherein the wearable alcohol sensor comprises a fuel cell module that generates electricity in the presence of alcohol.

4. The system of claim 1, wherein the processor is configured to display a user's past blood alcohol content for the user.

5. The system of claim 1, wherein the processor is configured to make predictions of a user's future blood alcohol content based on data received from the wearable alcohol sensor and display the predictions for the user.

6. The system of claim 1, wherein the processor is configured to warn a user when the user's blood alcohol content reaches a preset maximum value.

7. The system of claim 6, wherein the processor is configured to warn a user by at least one of sounding an alarm, causing the mobile device to vibrate, sending a text message, or initiating a phone call.

8. The system of claim 1, wherein the processor is configured to warn other people when a user's blood alcohol content reaches a preset maximum value.

9. The system of claim 1, wherein the mobile device further comprises a camera, wherein the processor is configured to take a picture of a user's surroundings if the user's blood alcohol content reaches a preset maximum value.

10. The system of claim 8, wherein the processor is configured to send the picture to at least one person other than the user.

11. The system of claim 1, wherein the processor is configured to predict the decrease in a user's blood alcohol content if the user does not ingest any further alcohol.

12. The system of claim 1, wherein the processor is configured to predict a time at which a user will be able to legally drive a motor vehicle if the user does not ingest any further alcohol.

13. The system of claim 12, wherein the processor is configured to contact at least one other person if a user's blood alcohol content is such that the user will not be able to legally drive a motor vehicle the same day.

14. The system of claim 1, wherein the processor uses past blood alcohol content measurements to calculate a user's rate of alcohol metabolism.

15. The system of claim 1, wherein the processor uses at least one of a user's height, weight, gender, and race to predict a user's rate of alcohol metabolism.

16. The system of claim 1, wherein the memory comprises a database, said database comprising information about the alcohol content of at least one alcoholic drink, wherein the processor is configured to use information from the database to make predictions about a user's blood alcohol content if the at least one alcoholic drink is consumed.

17. The system of claim 1, wherein the wearable sensor module comprises a secondary sensor, said secondary sensor capable of determining when an alcoholic drink is ingested by a user.

18. The system of claim 1, wherein the processor is configured to allow a user to manually enter information regarding a consumed alcoholic drink into the mobile device.

19. A method, comprising:
continuously measuring a user's blood alcohol content by a wearable sensor;
transmitting data from the wearable sensor to a mobile device;
analyzing the data by the mobile device;
using a result of the analysis to predict the user's future blood alcohol content.
